(19) United States
(12) Patent Application Publication
(10) Pub. No.: US 2005/0065424 A1
(43) Pub. Date: Mar. 24, 2005

(54) Method and system for volumemetric navigation supporting radiological reading in medical imaging systems

(75) Inventors: Deval V. Shah, St. Petersburg, FL (US); Steven L. Fors, Chicago, IL (US)

Correspondence Address: Lesavich High-Tech Law Group, P.C., Suite 325, 39 S. LaSalle Street, Chicago, IL, 60603, US

(73) Assignee: GE Medical Systems Information Technologies, Inc., Milwaukee, WI (US)

(21) Appl. No.: 10/861,781

(22) Filed: Jun. 4, 2004

Related U.S. Application Data
(60) Provisional application No. 60/476,757, filed on Jun. 6, 2003.

(51) Int. Cl. A61B 5/05; G06F 17/00

(52) U.S. Cl. 600/407; 128/922; 382/128

(57) ABSTRACT

A method and system for three-dimensional (3D) volumetric navigation supporting radiological readings in two-dimensional (2D) medical image display systems. Reformatted 3D medical image data such as magnetic resonance (MR) data is linked to raw and formatted 2D image data such as computed tomography (CT) data. An automatic synchronized navigation through entire linked 3D and 2D data sets is provided.

### CROSS REFERENCES TO RELATED APPLICATIONS

This patent application claims priority to U.S. Provisional Patent Application, 60/476,757, filed on Jun. 6, 2003, the contents of which are incorporated by reference.

### FIELD OF THE INVENTION

This invention relates to medical diagnostic systems. More specifically, it relates to a method and system for volumetric navigation supporting radiological readings in medical imaging systems.

### BACKGROUND OF THE INVENTION

In a modem radiology department, it is often desirable to view three-dimensional (3D) volumetric views and multi-planar reconstructions of patient information in addition to the two-dimensional (2D) raw images acquired during a computed tomography (CT) or magnetic resonance (MR) scan.

As is known in the medical arts, a “CT scan” is a radiographic technique that assimilates multiple X-ray images into a 2D cross-sectional image. An “MR scan” is a technique that uses the influence of a large magnet to polarize hydrogen atoms in the tissues and then monitors the summation of the spinning energies within living cells. It is used to produce 3D images of internal structures of the body, particularly the soft tissues.

These 3D visualizations are typically derived from a base series of 2D images acquired from a method of treatment or “modality” (e.g., CT or MR).

Typically only 2D base images are permanently stored on a Picture Archive and Communication System (PACS). The 3D images are generated when needed. As is known in the art, PACS is a computer and network system used by hospitals and clinics for digitized radiologic images and reports.

One problem with viewing 3D images is that the PACS system is capable of displaying original 2D base image sets, but the dynamic generation and display of the derived 3D images are typically relegated to a specialized workstation application tuned for 3D volumetric visualization. This separation of the 2D and 3D images makes it awkward for the radiologist to correlate what (s)he is viewing on the 3D visualization application with the raw 2D image date displayed on the PACS workstation.

Another problem is that there is no easy way to integrate and synchronize spatial context information between separate 2D and 3D viewing applications when viewing the same medical diagnostic image study.

Another problem with viewing 3D images on the PACS system is that it is often difficult to navigate through the large amount of data produced. Another problem is that as the amount of data increases, the time it takes to read and process the data to generate and view 3D images on the PACS system increases. Slow processing time may lead to frustrations by radiologists and may lead to increased diagnostic errors.

Thus it is desirable to enable a radiologist to view all data in a 3D volume-rendered state quickly. It is also desirable to enable a radiologist to navigate through this data in various display states (e.g., VR, MIP, MPR, etc.) on the fly. It is also desirable to link reformatted 3D data to raw and formatted 2D data in a PACS system.

### SUMMARY OF THE INVENTION

In accordance with preferred embodiments of the invention, some of the problems associated viewing different types of medical diagnostic images with are overcome. A method and system for volumetric navigation supporting radiological readings in medical image display systems is presented.

The method and system may provide 3D volumetric navigation supporting radiological readings in 2D medical image display systems. Reformatted 3D medical image data such as MR data is linked to raw and formatted 2D image data such as CT data. An automatic synchronized navigation through entire linked 3D and 2D data sets is provided.

The foregoing and other features and advantages of preferred embodiments of the invention will be more readily apparent from the following detailed description.

The detailed description proceeds with references to the accompanying drawings.

### BRIEF DESCRIPTION OF THE DRAWINGS

Preferred embodiments of the invention are described with reference to the following drawings, wherein:

FIG. 1 is a block diagram illustrating an exemplary medical diagnostic image system;

FIG. 2 is a block diagram illustrating exemplary medical image data linking;

FIG. 3 is a block diagram illustrating an exemplary medical image data linking architecture;

FIG. 4 is a flow diagram illustrating a method for linking points on different medical images;

FIG. 5 is a block diagram illustrating an exemplary medical image object architecture;

FIG. 6 is a data flow diagram illustrating details of the medical image object architecture;

FIG. 7 is a flow diagram illustrating a method for displaying a medical image;

FIG. 8 is a block diagram illustrating screen shoots of a control panel used to initiate the invention; and

FIG. 9 is a block diagram illustrating screen shoots illustrating diagnostic image viewing according to the invention.

### DETAILED DESCRIPTION OF THE INVENTION

Exemplary Medical Diagnostic Image System

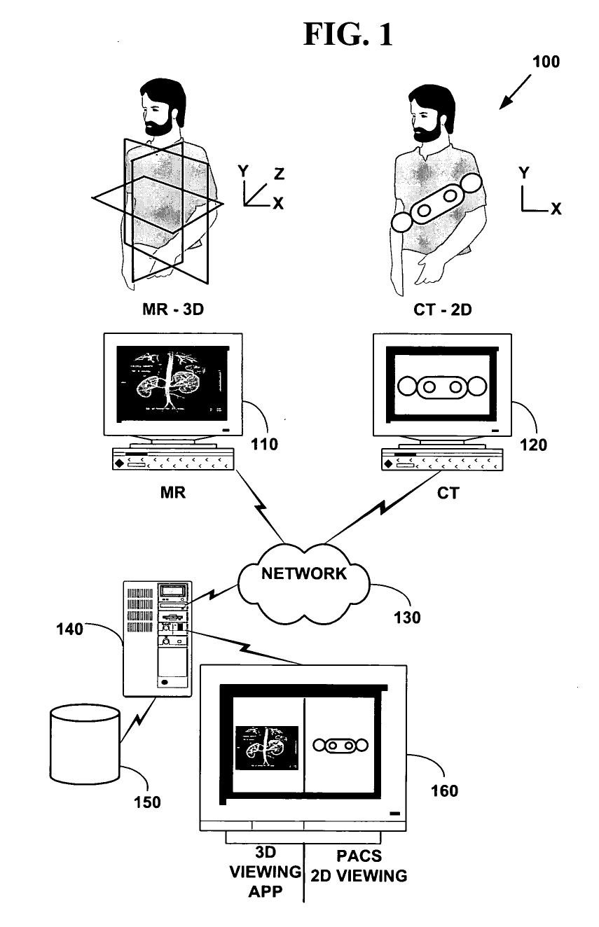

FIG. 1 is a block diagram illustrating an exemplary medical diagnostic image system 100. The medical diagnostic image system 100 includes, but is not limited to, an 3D medical image system 110 (e.g., MR, etc.), a 2D medical image system 120 (e.g., CT, etc.) a communications network 130, one or more server computers 140, one more databases 150 and one or more display devices includes graphical viewing terminals 160. However, more or fewer components can also be used in the medical diagnostic image system 100 and the invention is not limited to these components. The medical diagnostic image system 100 may comprise a portion of a PACS system.

An operating environment for components of the medical diagnostic image system 100 include a processing system with one or more high speed Central Processing Unit(s) (“CPU”), processors and one or more memories. In accordance with the practices of persons skilled in the art of computer programming, the invention is described below with reference to acts and symbolic representations of operations or instructions that are performed by the processing system, unless indicated otherwise. Such acts and operations or instructions are referred to as being “computer-executed,” “CPU-executed,” or “processor-executed.”

It will be appreciated that acts and symbolically represented operations or instructions include the manipulation of electrical signals by the CPU or processor.

An electrical system represents data bits which cause a resulting transformation or reduction of the electrical signals and the maintenance of data bits at memory locations in a memory system to thereby reconfigure or otherwise alter the CPU's or processor's operation, as well as other processing of signals. The memory locations where data bits are maintained are physical locations that have particular electrical, magnetic, optical, or organic properties corresponding to the data bits.

The data bits may also be maintained on a computer readable medium including magnetic disks, optical disks, organic memory, and any other volatile (e.g., Random Access Memory (“RAM”)) or non-volatile (e.g., Read-Only Memory (“ROM”), flash memory, etc.) mass storage system readable by the CPU. The computer readable medium includes cooperating or interconnected computer readable medium, which exist exclusively on the processing system or can be distributed among multiple interconnected processing systems that may be local or remote to the processing system.

Exemplary Location Information Cursor Linking

In one embodiment of the invention, 3D position information is integrated between 2D and 3D viewing paradigms by providing a mechanism called “3D cursor.” However, the 3D cursor name is a moniker only and other moniker can also be used to identify features of the invention. The 3D cursor mechanism provides communication between the 2D and 3D viewing paradigm systems. The 3D cursor mechanism allows for immediate synchronized navigation through different image sets while they are being simultaneously viewed on the two different applications (e.g., the 2D PACS and a 3D visualization application).

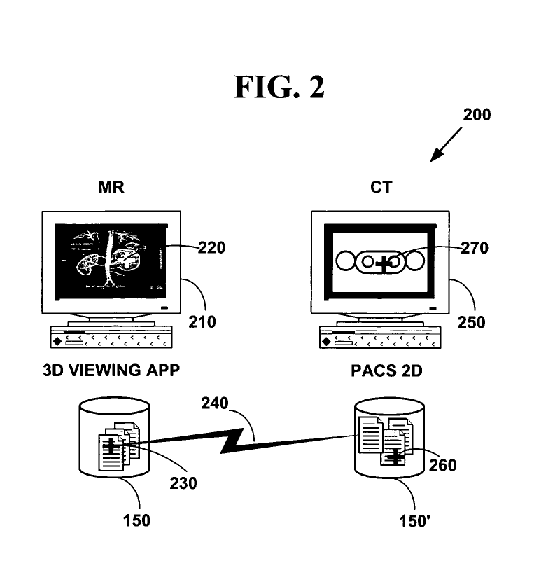

FIG. 2 is a block diagram illustrating exemplary medical image data linking 200. When a radiologist manipulates a 3D visualization application 210 (e.g., to display sagittal and coronal reformat images) and a point in the 3D space of the patient information is identified via a cross-hair 220 or other mechanism on an image 230, this exact point location will be transmitted 240 to a 2D viewing application such as a PACS system 250 via a standardized format based on the patient coordinate system.

FIG. 2 illustrates the 3D 210 and 2D 250 viewing applications on separate graphical terminals. However, the invention is not limited to such an embodiment.

However, these viewing applications may appear on the same display device (i.e., graphical terminal 160) in separate portions of the viewing screen (e.g., separate windows).

In computed tomography, when data from a series of contiguous transverse scan images are made along an axis of a plane or “axial” plane are recombined to produce images in a different plane, such as a sagittal or coronal plane, the images are called “reformat” images. A “sagittal plane” is a median plane of a body dividing the body into left and right halves. A “coronal plane” is a vertical plane through a body from head to foot and parallel to the shoulders, dividing the body into front and back halves.

When the PACS system 250 receives this 3D point via the 3D cursor method it will identify the appropriate image 260 within the base set that contains the point, automatically navigate to that slice, and identify that same point with a marker 270 (cross-hair, etc.). This will occur virtually immediately during simultaneous display of the study within the two viewing applications (210, 250).

The 3D position, or “cursor” is thus linked bidirectionally across the two viewing applications 210, 250, facilitating the correlation of views between them.

Similarly, when the radiologist navigates through the base 2D images 260 on the PACS system 250, appropriate 3D cursor information will be transmitted 240 to the 3D visualization application 210 and the appropriate 3D image 230 at the point marker 220.

Exemplary Location Information Linking Architecture

One exemplary architecture for the 3D cursor includes a mechanism for communicating a location of a 3D cursor within a given Digital Imaging and Communications in Medicine (DICOM) series amongst arbitrary software components. However, the present invention is not limited to this embodiment and other embodiments can also be used to practice the invention.

The DICOM standard specifies a hardware independent network protocol utilizing Transmission Control Protocol (TCP) over Internet Protocol (IP) and defines operation of Service Classes beyond the simple transfer of data. It also creates a mechanism for uniquely identifying Information Objects as they are acted upon across a network. DICOM defines Information Objects not only for images but also for patients, studies, reports, and other data groupings. The National Electrical Manufacturers Association (NEMA) DICOM PS 3-2003 standard is incorporated herein by reference.

When the 3D cursor position is directly modified in one of these arbitrary software components, all other components immediately update their views to indicate the new cursor position. 3D cursor synchronization is bidirectional so that a user is free to switch between components, modifying a cursor position from any of them, and having the respective passive components automatically reflect the change in state. No component shall have exclusive “mastership” of the cursor. Any number of software components can participate in 3D cursor sharing. 3D Cursor change events are broadcast to all but the originator of the event.

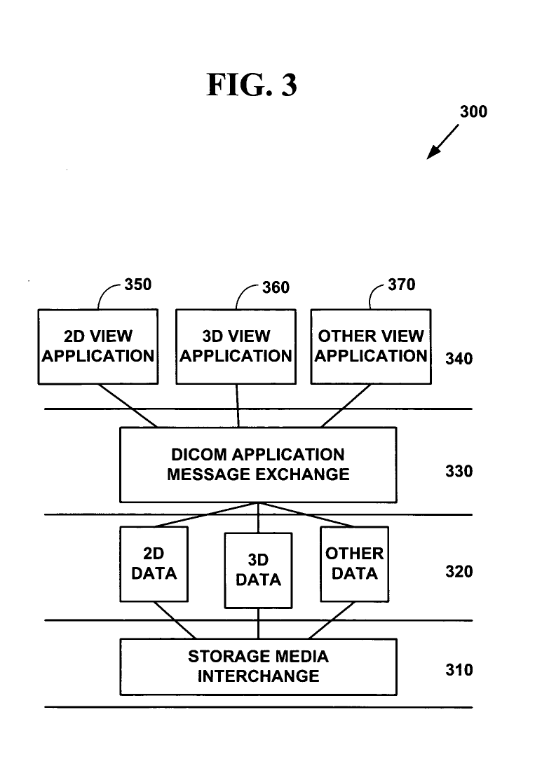

FIG. 3 is a block diagram illustrating an exemplary medical image data linking architecture 300. The 3D cursor linking architecture includes, but is not limited to, a storage media layer interchange layer 310, a storage media layer 320 including 2D data, 3D data, and other types of data (e.g., text, etc.), a DICOM message exchange layer 330, and a viewing application layer 340 including a 2D viewing application 350 (e.g., PACS), a 3D viewing application 360 and other types of viewing applications 370 (e.g., text, etc.). However, the present invention is not limited to this embodiment and other embodiments can also be used to practice the invention.

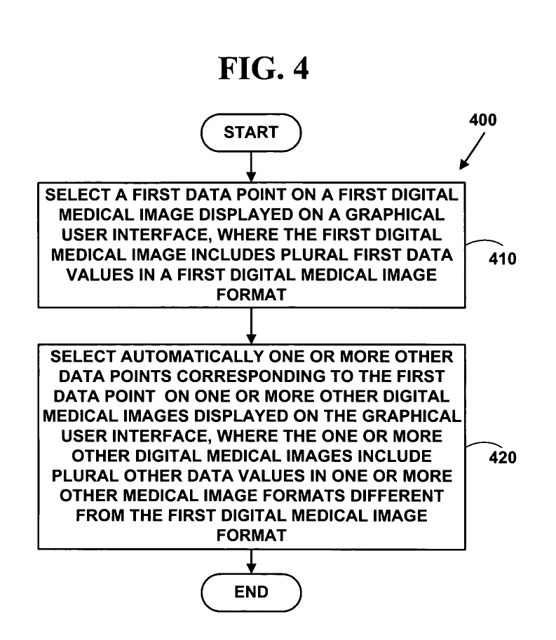

FIG. 4 is a flow diagram illustrating a Method 400 for linking points on different medical images. At Step 410, a first data point on a first medical image is selected on a display device. The first digital medical image includes plural first data values in a first digital medical image format. At Step 420, one or more other data points corresponding to the first data point are automatically selected on one more other digital medical images displayed on the display device. The one or more other digital medical images include plural other data values in one or more other digital medical image formats different from the first digital medical image format.

Method 400 provides bidirectionally across the two viewing applications facilitating the correlation of views between them. In one embodiment of the invention, the first data point is a 3D data point 220 on an MR image and the one or more other data points are 2D data points 270 on a CT image. In another embodiment of the invention, the first data point is a 2D data point 270 on a CT image and the one or more other data points are one or more 3D data points 220 on an MR image.

In one embodiment of the invention, Method 400 is executed on a PACS system. In another embodiment of the invention, Method 400 is executed on a DICOM system. However, the present invention is not limited to these embodiments and other embodiments can also be used to practice the invention.

One embodiment of the invention includes a medical imaging system with a position information module for providing position information for two different medical viewing formats simultaneously to two different medical viewing applications and a bidirectional communications module for providing bi-direction communications of position information from the position information module between the two different medical viewing applications, thereby allowing for immediate synchronized navigation through different medical image sets from the two different medical viewing formats while they are being simultaneously viewed on the two different medical viewing applications. However, the present invention is not limited to this embodiment and other embodiments can also be used to practice the invention.

Exemplary Location Information Object Architecture

In one exemplary embodiment of the invention, the 3D cursor and Method 400 is implemented using object-oriented objects. However, the invention is not limited to such an embodiment, and other object-oriented and other non-object oriented implementations can also be used.

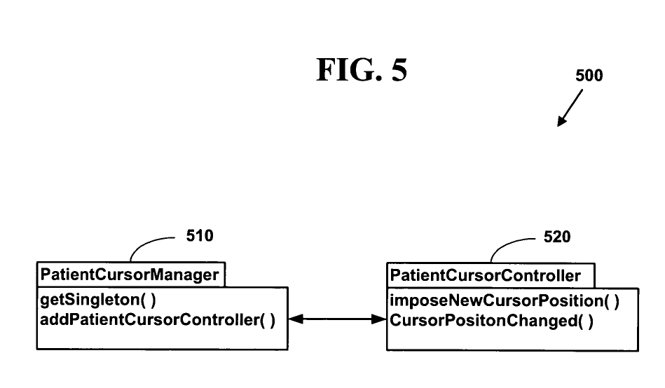

FIG. 5 is a block diagram illustrating an exemplary medical image linking object architecture 500.

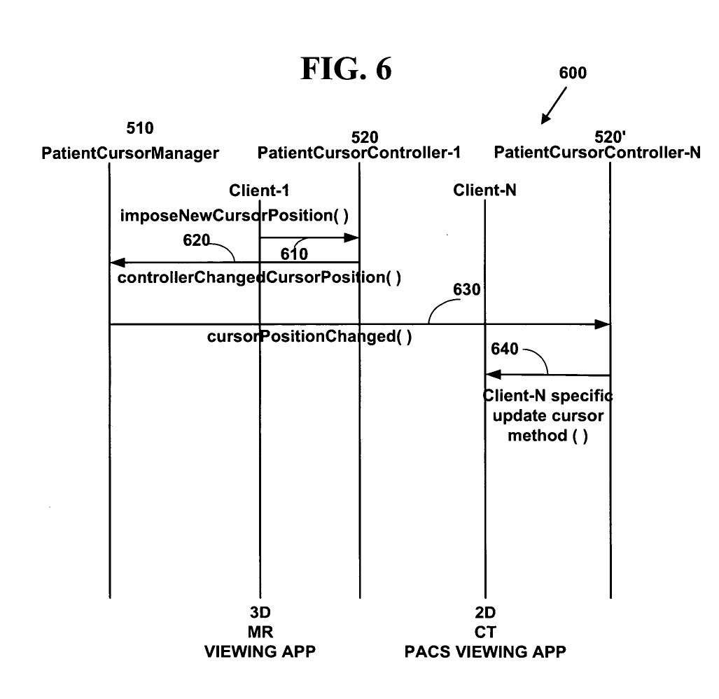

FIG. 6 is a data flow diagram 600 illustrating details of the medical image linking object architecture 500 of FIG. 5.

In one embodiment of the invention, the 3D cursor communication is based on communication between two object-oriented objects called “PatientCursorManager( )” 510 and “PatientCursorController( )” 520 However, the invention is not limited to such an embodiment and the 3D cursor communication can be based on other object-oriented objects, or other non-object-oriented objects.

In one embodiment of the invention, the 3D cursor communication is used in a PACS system. In another embodiment of the invention, 3D cursor communication is used on a DICOM system. However, the present invention is not limited to these embodiments and other embodiments can also be used to practice the invention.

These objects interact in a Mediator pattern with PatientCursorManager() 510 acting as the Mediator to PatientCursorController( ) 520, which are Colleagues in the Mediator Pattern. Clients add PatientCursorControllers( ) 520 to the singleton PatientCursorManager( ) 510. The PatientCursorManager( ) 510 mediates the 3D cursor position changes amongst all PatientCursorControllers( ) 520.

In one embodiment of the invention, there is no direct interaction amongst the PatientCursorControllers( ) 520. In another embodiment of the invention, there is direct interaction amongst the PatientCursorControllers( ) 520.

The PatientCursorController( ) 520 is modeled as a Singleton pattern. PatientCursorController( ) 520 is an object base class that provides an interface for components that wish to participate in 3D cursor sharing. In order to participate in 3D cursor sharing, a client component defines a subclass of PatientCursorController() 520 and registers an instance of it with the PatientCursorManager( ) 510.

The PatientCursorController( ) 520 implements a “cursorPositionChanged( )=38 object method to react to changes to the 3D cursor. This in an input method, and is called by the PatientCursorManager( ) 510 to communicate new cursor information when appropriate to each of the PatientCursorControllers() 520. When called, the derived PatientCursorController( ) 520 subclass updates its view to indicate the new 3D cursor location. This update includes moving the 3D cursor on the rendered image, or requires navigation to the correct digital image slice where the indicated location can be found.

Another object method, “imposeNewCursorPosition( ),” is provided by the base class implementation of PatientCursorController( ) 520. This method is called whenever any particular PatientCursorController( ) 520 wants to impose a new 3D cursor.

For example, returning to FIG. 6, Client-1 (e.g., a 3D MR viewing application) imposes a new 3D cursor position by calling 610 imposeNewCursorPosition( ). The corresponding PatientCursorController( ) 520 for Client-1 calls 620 controllerChangedCursorPosition( ) in the PatientCursorManger( ) 510. The PatientCursorManager( ) 510 calls 630 cursorPositionChanged( ) in the PatientCursorController() 520′ for Client-N (e.g., 2D CT PACS viewing application). PatientCursorController( ) 520′ for Client-N calls 640 a Client-N specific cursor update method to update a cursor position provided by the 3D cursor information.

In cases where the 3D cursor needs to be synchronized with another application or remote application, the “client” code takes the form of an object adapter that communicates with the external code. This communication based on the communication scheme used by the selected application. For example, the communication includes Component Object Model (COM), Common Object Request Broker Architecture (CORBA), Window's messaging, or other types of communication.

For example, suppose there is an external application called “AW”. A CORBA remote method interface is defined for passing 3D cursor information (e.g., “sendPoint”). Within the Centricity application, the adapter class for the AW client application (e.g., 3D MR viewer) creates a PatientCursorController( ) 520 that would field any “sendPoint” calls generated by AW, and relay these to all other PatientCursorControllers( ) 520 via the PatientCursorManager( ) 510 by calling “imposeNewCursorPosition( )”. Likewise, this AW adapter PatientCursorController 520 would implement its “cursorPositionChanged( )” method by calling “sendPoint” on the remote AW object.

Note that the above example is only one of many plural possible implementations of the invention. However, the invention is not limited to such an implementation and other implementations can also be used.

Each component and adapter is free to use entirely separate and different mechanisms for the low-level communication. Building on the above example, say another external component “TR” required 3D cursor synchronization, but the only way to communicate this info was via simple “sockets” packets. An adapter and PatientCursorController 520( ) are built for this mechanism.

Although the 3D cursor object architecture 500 is unaware of these separate mechanisms, the loose coupling provides a mechanism whereby the 3D cursor information received from AW via the CORBA “sendPoint” method is automatically translated into the appropriate socket data packet and sent to TR. AW and TR can both participate in the 3D cursor synchronization, whether or not Centricity itself is doing so.

Exemplary Location Information Data Interfaces

In one embodiment of the invention, 3D cursor position data is communicated via arguments in the imposeNewCursorPosition( ) and cursorPositionChanged( ) objects methods. However, the invention is not limited to this embodiment and other object-oriented and non-object oriented embodiments can also be used to communicate 3D cursor data.

Table 1 illustrates exemplary Java object method interfaces that define exemplary 3D cursor position data interfaces used in the 3D cursor object architecture 500. However, the invention is not limited to these Java object method interfaces and other types of interfaces can also be used (i.e., other object-oriented and non-objected-oriented interfaces).

TABLE 1
public final void imposeNewCursorPosition(String seriesInstanceUID,
Point3d threeDPoint);
public void cursorPositionChanged(String seriesInstanceUID,
Point3d threeDPoint);
Copyright © 2003 by GE Medical Systems, Inc.
All rights reserved.

The “Point3d threeDPoint” argument is an object class available in the Java3d “vecmath.jar” library, the contents of which are incorporated by reference. Point3D threeDPoint encapsulates an ordered triplet (x,y,z) of three double values.

This argument codes the 3D cursor position in the coordinate space defined by the Image Position and ImageOrientation fields found in DICOM 330 image headers. As long as all client components are aware of this point of reference (which is a well-defined DICOM standard) the positions are coherent amongst them.

The argument “String serieslnstanceUID” provides the DICOM series instance UID for the cursor change. It is assumed that the images in a DICOM series define a 3D “voxel” volumetric space. The 3d position is an (x,y,z) position within this voxel space. The series instance UID is therefore used to communicate which volume the 3D cursor change applies. This allows for a separate 3D cursor for every series.

It is common for applications to be able to display more than one image series at a time, so this information is used for matching up the image correct series.

Since the 3D cursor object architecture 500 broadcasts 3D cursor changes to controllers 520 (there is no automatic filtering in the event delivery), each PatientCursorController( ) 520 validates the UID when cursorPositionChanged( ) is called.

The 3D cursor object architecture 500 also provides a way to designate that certain PatientCursorControllers( ) 520 are only interested in 3D cursor changes for specific UIDs.

The method and system described herein provides the integration of at least two distinct viewing paradigms (e.g., 2D and 3D) by providing a standardized mechanism for the communication of 3-dimensional position information via 3D Cursor, between them. This allows for immediate synchronized navigation through the digital image sets while simultaneously viewed on the two applications (e.g., PACS and 3D visualization applications).

However, the method and system described herein is not limited to 2D and 3D viewing paradigms and can also be used to link other medical image diagnostic information together (e.g., text, etc.).

Exemplary Volumetric Navigational Viewing

The method and system described herein provides the integration of at least two distinct viewing paradigms (e.g., 2D and 3D) by providing a standardized mechanism for the communication of 3-dimensional position information via 3D Cursor, between them. This allows for immediate synchronized navigation through the digital image sets while simultaneously viewed on the two applications (e.g., PACS and 3D visualization applications).

However, the method and system described herein is not limited to 2D and 3D viewing paradigms and can also be used to link other medical image diagnostic information together (e.g., text, etc.).

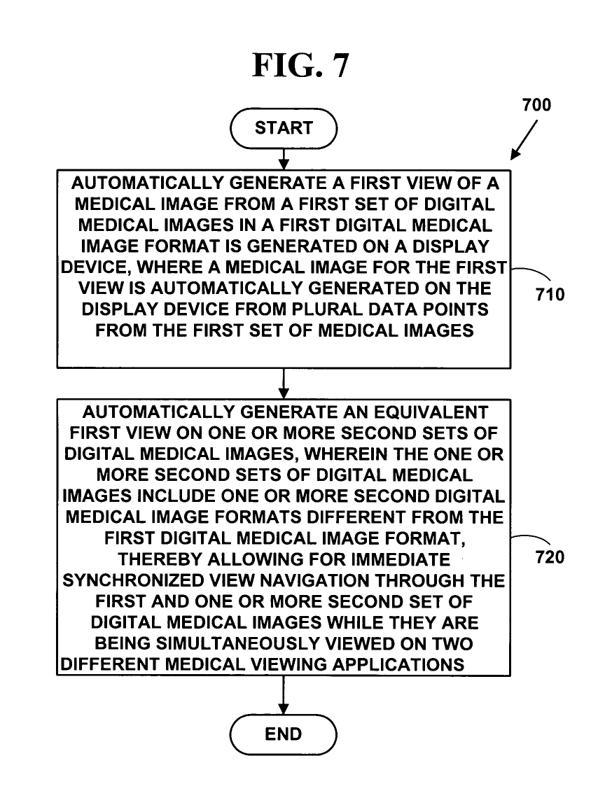

FIG. 7 is a flow diagram illustrating a Method 700 for displaying a medical image. At Step 710, a first view of a medical image from a first set of digital medical images in a first digital medical image format is automatically generated on a display device. A medical image for the first view is automatically generated on the display device from plural data points from the first set of medical images. At Step 720, an equivalent second view on one or more second sets of digital medical images is automatically generated. The one or more second sets of digital medical images include one or more second digital medical image formats different from the first digital medical image format, thereby allowing for immediate synchronized view navigation through the first and one or more second set of digital medical images while they are being simultaneously viewed on two different medical viewing applications.

Method 700 enables a radiologist to view data in a 3D volume-rendered state quickly. It is also enables a radiologist to navigate through this data in various display states (e.g., VR, MIP, MPR, etc.) on the fly. It also links reformatted 3D data to raw or formatted 2D data in a PACS system, DICOM system, or other medical image display system.

Method 700 also allows for 3D volumetric navigation supporting radiological readings in two-dimensional 2D medical image display systems.

Reformatted 3D medical image data is automatically linked to raw and formatted 2D image data. A synchronized navigation through entire linked 3D and 2D data sets is thereby provided.

In one embodiment of the invention, Step 720 includes execution of Method 400 of FIG. 4. However, the present invention is not limited to this embodiment and the present invention can be practiced without executing Method 400 at Step 720.

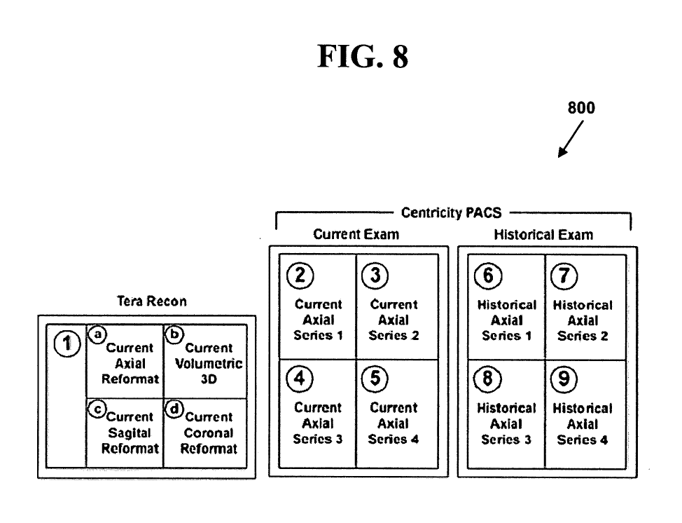

FIG. 8 is a block diagram illustrating screen shoots 800 of a graphical control panel used to initiate one exemplary embodiment invention. The viewports mentioned below in Tables 2-3 are illustrated in FIG. 8. The graphical control panel buttons are used at Step 720 to automatically select new views of medical images.

However, the present invention is not limited to use with this control panel and can be used in medical imaging systems with a different control panel or automatically without any control panel.

One embodiment of the invention includes a medical image viewing system comprising a viewing module for providing viewing information for two different medical viewing formats simultaneously for two different medical viewing applications; and a bidirectional communications module for providing bi-direction communications of position information from the viewing module between the two different medical viewing applications, thereby allowing for immediate synchronized navigation through different medical image sets from the two different medical viewing formats while they are being simultaneously viewed on the two different medical viewing applications. However, the present invention is not limited to this embodiment and other embodiments can also be used to practice the invention.

In one embodiment of the invention, the viewing module and bidirectional communications module use the object-oriented objects described in association with FIGS. 5 and 6 above. However, the present invention is not limited to this embodiment and other embodiments, including other object-oriented objects and other non-object-oriented objects can also be used to practice the invention.

Table 2 illustrates exemplary details of installing Method 700 in a 3D image viewing application in a medical imaging system. However, the present invention is not limited to the details illustrated in Table 2 and other details can also be used to practice the invention. The references to Centricity include the GE Medical Systems Centricity™ medical viewing applications. The Centricity applications were developed in collaboration with doctors to meet the specific requirements of radiologists, hospital doctors and nursing staff. However, the present invention is not limited to the GE Centricity applications and the present invention can be used in other medical imaging systems with other medical imaging software.

```
TABLE 2
Installation of 3D Application on a 3D viewing workstation 210:
    Layout, monitor information gets registered (entered) during installation. 3D application is
    wrapped in an executable and able to communicate to Centricity using COM (JIntegra),
    CORBA (visibroker) or CORBA (java).
    The installation will take care of 3D Application server, 3D specific user security
    information and should be transparent to Centricity.
    Server connection establishment and exceptions are handled by 3D Application client
    and Centricity does not have to manage
    3D Application provides an installation script taking care of the above.
Login:
    Upon Centricity login, user information (username, password, domain) are passed to 3D
    application to establish the secured session to 3D application Server.
Logout/Quit:
    Upon Centricity logout/quit, 3D Application appropriately logout/quits and releases
    whatever needed to be released on 3D application server (e.g. session, memory)
Opening of an exam in Centricity:
    Seamless integration between Centricity and 3D application is provided. The radiologists'
    workflow can be driven from either side (Centricity as well as 3D Application). The user
    can switch exam or series from either of the applications.
    Patient information (study instance uids, patient name, series instance uids, protocol, etc)
    is automatically passed to 3D application depending upon 3D DDPs defined in Centricity
    DDP wizard. Predefined 3D protocols (per exam procedure) are passed instead of
    possibly entire 3D DDP XML stream.
    3D Application checks the following and takes action depending upon the following.
    (1) Same StudyInstance uid, Same Series instance uid, same protocol: Do nothing,
        Bring the 3D application to front. The exam is not reloaded.
    (2) Same Study Instance uid, Same Series instance uid, different protocol: Replace the
        protocol on an already opened exam. The exam is not reloaded from scratch.
    (3) Same Study Instance uid, Different Series Instance uid, same/different protocol:
        Replace the loaded series with a different series. The exam is not reloaded from
        scatch.
    (4) Different exam (i.e. different studyinstance uid): Close all the previously opened
        exams and reload the new one with the given series and protocol.
    Close the previously opened exam (if it cannot have more than one open at the same
    time) and open the new one.
    For CR/DX exams for which 3D is not needed, “CloseExam” is called. Enabling 3D
    Application to close the exam and follow the same sequence as in “CloseExam”. There is
    not reference of the exam (in patient list etc) should be there for the user to select.
    The user can perform the advanced 3D analysis (what is not available already on 2D
    PACS) by working entirely in 3D Application on mixed monitor.
    Patient, exam and series level context need to be maintained between 3D Application
    and Centricity.
    In case of Current and Historical, both exam Ids are passed to 3D application as above.
    Depending upon the DDP, all series UIDs for the exams on Centricity Image viewing area
    is passed to 3D application. The 3D application will display Current and Historical 3D
    models the exams.
Closing of an exam in Centricity:
    3D Application closes (without opening a new one) the opened study and hide itself
    bringing Centricity pallettes up on the mixed monitor.
Applying Centricity tools in Centricity: Window/Level valued are shared back and forth
between Centricity and 3D application.
Hide/Show 3D Application:
    Brings 3D Application (PACS exam already loaded in 3D Application) to focus on the
    mixed monitor.
    Depending upon 3D application configuration, the 3D application is either embedded in
    Centricity (preferably on the mixed monitor) or invoked from Centricity depending upon
    exam open request or 3D button press on the Centricity Image viewing area.
Manual loading of 3D Application:
    Manual launching of 3D Application for cases where relevant 3D protocols are not
    defined for automatic loading. (A button somewhere in Centricity, similarly, on 3D
    Application)
Linked Cursor (Integrate Navigational systems): 3D Cursor is developed in Centricity. Cursor
position coordinates (series instance uids, x, y and z coordinates) are passed back and forth.
Linked Series: Linked series is a prerequisite for Linked cursor functionality. If a series is
changed in Centricity for an exam and 3D application is invoked (by pressing 3D button) the 3D
application will show the correct series.
Defining DDP with 3D
Applying DDP with 3D
```

Table 3 illustrates exemplary details of using Method 700 in a 3D image viewing application in a medical imaging system. However, the present invention is not limited to the details illustrated in Table 3 and other details can also be used to practice the invention.

```
TABLE 3
First Workflow Example
    Anonymized Lung CT nodule exam
    Viewport 1a - Tera Recon Axial Reformat
    Viewport 1b - Tera Recon Volumetric 3D
    Viewport 1c - Tera Recon Sagital Reformat
    Viewport 1d - Tera Recon Coronal Reformat
    Viewport 2 - Current Axial Series 1 - Same series as displayed on 1a-1d
    Viewport 3-5 - Other axial series, perhaps scout if available
    Viewport 6-9 - Historical Axial Series -
    Viewport 1b - Volumetric 3D data displays by default with the protocol that will best allow a
    radiologist to locate and identify lung nodules.
        a. Radiologist will rotate, pan, zoom, subtract tissue, adjust window/level and
          transparency values to key in on a particular nodule
        b. Once the radiologist locates a particular nodule, he will move from the volumetric 3D
          reconstruction to Viewports 1a, 1c or 1d to view with better granularity
        c. Because Viewports 1a-1d are linked to Viewport 2 (PACS), the datasets update as
          the radiologist manipulates the data
    Viewport 1a, 1c, 1d - Axial, Sagittal and Coronal reformats. Radiologists will manipulate
    these data sets to better understand spatial relationships.
        a. Pan, zoom rotate to better position the data
        b. Window/level, transparency and slice thickness is adjusted to better view the data
        c. Radiologists will also view the data at oblique angles
        d. Once the radiologist pinpoints the nodule in Viewports 1a-1d, he will go to viewport
          2 to confirm. In the full application, Viewport 2 is linked to Viewports 1a-1d, so as the
          data sets are manipulated, Viewport 2 will update in synchronization.
    Viewport 2 - The data will automatically synchronize to the approximate location within
    Viewports 1a-1d.
        a. Radiologist will manually cine to confirm with the raw data
        b. Full PACS imaging tools are available
        c. Radiologist may measure and annotate on the raw data
    At this point, the radiologist can conclude this exam by reporting and closing the exam to
    open the next exam.
Second Workflow Example - Aneurysm
    Anonymized Abdominal Aneurysm Exam
    Viewport 1a - Tera Recon Axial Reformat
    Viewport 1b - Tera Recon Volumetric 3D
    Viewport 1c - Tera Recon Sagital Reformat
    Viewport 1d - Tera Recon Coronal Reformat
    Viewport 2 - Current Axial Series 1 - Same series as displayed on 1a-1d
    Viewport 3-5 - Other axial series, perhaps scout if available
    Viewport 6-9 - Historical Axial Series -
    Viewport 1b - Volumetric 3D data displays by default with the protocol that will best allow a
    radiologist to locate and identify lung nodules.
        a. Radiologist will rotate, pan, zoom, subtract tissue, adjust window/level and
          transparency values to key in on a particular nodule
        b. Once the radiologist locates a particular nodule, he will move from the volumetric 3D
          reconstruction to Viewports 1a, 1c or 1d to view with better granularity
        c. Because Viewports 1a-1d are linked to Viewport 2 (PACS), the datasets update as
          the radiologist manipulates the data
    Viewport 1a, 1c, 1d - Axial, Sagittal and Coronal reformats. Radiologists will manipulate
    these data sets to better understand spatial relationships.
        a. Pan, zoom rotate to better position the data
        b. Window/level, transparency and slice thickness are adjusted to better view the data
        c. Radiologists will also view the data at oblique angles
        d. Once the radiologist pinpoints the nodule in Viewports 1a-1d, he will go to viewport
          2 to confirm. In the full application, Viewport 2 is linked to Viewports 1a-1d, so as the
          data sets are manipulated, Viewport 2 will update in synchronization.
    Viewport 2 - In the data will automatically synchronize to the location within Viewports 1a-1d.
        a. Radiologist will manually cine to confirm with the raw data
        b. Full PACS imaging tools are available
        c. Radiologist may measure and annotate on the raw data
    At this point, the radiologist can conclude this exam by reporting and closing the exam to
    open the next exam.
```

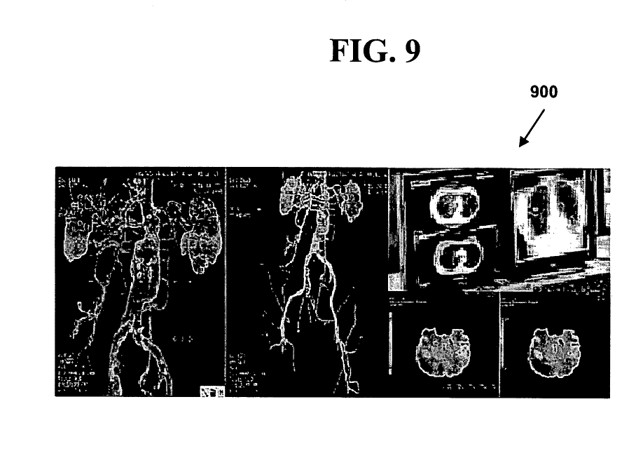

FIG. 9 is a block diagram illustrating screen shoots 900 illustrating diagnostic image viewing according to the invention generated with selecting viewports of FIG. 8.

It should be understood that the architecture, programs, processes, methods and systems described herein are not related or limited to any particular type of computer or network system (hardware or software), unless indicated otherwise. Various types of general purpose or specialized computer systems may be used with or perform operations in accordance with the teachings described herein.

While various elements of the preferred embodiments have been described as being implemented in software, in other embodiments hardware or firmware implementations may alternatively be used, and vice-versa.

In view of the wide variety of embodiments to which the principles of the invention can be applied, it should be understood that the illustrated embodiments are exemplary only, and should not be taken as limiting the scope of the invention. For example, the steps of the flow diagrams may be taken in sequences other than those described, and more or fewer elements may be used in the block diagrams.

The claims should not be read as limited to the described order or elements unless stated to that effect. In addition, use of the term “means” in any claim is intended to invoke 35 U.S.C. §112, paragraph 6, and any claim without the word “means” is not so intended. Therefore, all embodiments that come within the scope and spirit of the following claims and equivalents thereto are claimed as the invention.

1. A method for viewing medical images, comprising:
automatically generating a first view of a medical image on a display device from a first set of digital medical images in a first digital medical image format, wherein the medical image for the first view is automatically generated on the display device from a plurality of data points from the first set of medical images; and
automatically generating an equivalent second view on one or more second sets of digital medical images, wherein the one or more second sets of digital medical images include one or more second digital medical image formats different from the first digital medical image format, thereby allowing for immediate synchronized view navigation through the first and one or more second set of digital medical images while they are being simultaneously viewed on two different medical viewing applications.

2. The method of claim 1 further comprising a computer readable medium having stored therein instructions for causing a processor to execute the steps of the method.

3. The method of claim 1 wherein the first digital medical image format includes a magnetic resonance image format and the one or more second digital medical image formats include computed tomography image formats.

4. The method of claim 1 wherein the first digital medical image format includes a plurality of three-dimensional points and the one or more second digital medical image formats includes a plurality of two-dimensional data points.

5. The method of claim 1 wherein the first digital medical image format includes a plurality of two-dimensional points and the one or more other digital medical image formats includes a plurality of three-dimensional data points.

6. The method of claim 1 wherein the method is executed on a Picture Archive and Communication System (PACS).

7. The method of claim 1 wherein the method is executed on a Digital Image and Communications in Medicine (DICOM) system.

8. The method of claim 1 wherein the one or more other second sets of digital medical images include a current second set of digital medical images and a historical second set of digital medical images.

9. The method of claim 1 wherein the step of automatically generating a first view includes automatically generating an axial, sagittal or coronal plane view.

10. The method of claim 1 wherein the step of automatically generating an equivalent second view includes automatically generating an axial, sagittal or coronal plane view.

11. The method of claim 1 wherein the first view and the equivalent second view are automatically generated using a plurality of object-oriented objects.

12. A medical imaging system, comprising:
a viewing means for providing viewing information for two different medical viewing formats simultaneously for two different medical viewing applications; and
a bidirectional communications means for providing bi-direction communications of position information from the viewing means between the two different medical viewing applications, thereby allowing for immediate synchronized navigation through different medical image sets from the two different medical viewing formats while they are being simultaneously viewed on the two different medical viewing applications.

13. The medical imaging system of claim 12 wherein the two different medical viewing formats include a three-dimensional medical viewing format and a two-dimensional medical viewing format.

14. The medical imaging system of claim 13 wherein the three-dimensional medical viewing format includes a magnetic resonance image format.

15. The medical imagines system of claim 13 wherein the two-dimensional medical viewing format includes a computed tomography image format.

16. The medical imaging system of claim 12 wherein the viewing means and the bidirectional communications means comprise one or more object-oriented objects.

17. The medical imaging system of claim 12 wherein the medical imaging system is included on a Picture Archive and Communication System (PACS).

18. The medical imaging system of claim 12 wherein the medical imaging system is included on a Digital Image and Communications in Medicine (DICOM) system.

19. A method for viewing medical images, comprising:
automatically generating a first three-dimensional view of a medical image on a display device from a first set of digital medical images in a first digital medical image format, wherein the medical image for the first three-dimensional view is automatically generated on the display device from a plurality of data points from the first set of medical images; and
automatically generating an equivalent first two-dimensional view on one or more second sets of digital medical images, wherein the one or more second sets of digital medical images include one or more second digital medical image formats different from the first digital medical image format, thereby allowing for immediate synchronized view navigation through the first and one or more second set of digital medical images while they are being simultaneously viewed on two different medical viewing applications.

20. The method of claim 19 further comprising a computer readable medium having stored therein instructions for causing a processor to execute the steps of the method.

21. The method of claim 19 wherein first three-dimensional view includes a magnetic resonance image view and the equivalent first two-dimensional view includes a computed tomography image view.
